(19) United States
(12) Patent Application Publication
(10) Pub. No.: US 2008/0058254 A1
(43) Pub. Date: Mar. 6, 2008

(54) Composition and method for enhancing or promoting the activity of insulin, enhancing skeletal muscle growth, reducing skeletal muscle loss, and increasing the energy supply to skeletal muscle

(76) Inventors: Marvin A. Heuer, Mississauga (CA); Shan Chaudhuri, Mississauga (CA); Kenneth Clement, Mississauga (CA)

Correspondence Address: KENYON & KENYON LLP, ONE BROADWAY, NEW YORK, NY, 10004, US

(21) Appl. No.: 11/514,750

(22) Filed: Aug. 31, 2006

(51) Int. Cl. A61K 38/22; A61K 31/716; A61K 31/385; A61K 31/185; A61K 31/198; A61K 31/045

(52) U.S. Cl. 514/12; 514/54; 514/561; 514/553; 514/729; 514/440

(57) ABSTRACT

A dietary supplement and method for enhancing or promoting the activity of insulin, enhancing skeletal muscle growth, reducing skeletal muscle loss, and increasing the energy supply to skeletal muscle of an individual comprising at least D-Pinitol and Leucine or derivatives thereof. The dietary supplement may further comprise at least one of Alpha Lipoic Acid, Glucomannan, D-Myo-lnositol, Guar Gum, Taurine or derivative thereof, a derivative of Ketoisocaproic Acid, a derivative of Alpha-Ketoglutarate, and a source of Peptide C12.

### FIELD OF THE INVENTION

The present invention relates to the composition of a dietary supplement and methods for enhancing or promoting the activity of insulin, enhancing skeletal muscle growth, reducing skeletal muscle loss, and increasing the energy supply to skeletal muscle.

### BACKGROUND OF THE INVENTION

The most common role associated with insulin is the carbohydrate-induced uptake of glucose by cells. The release of glucose from cells is also concomitantly inhibited by insulin and its storage as glycogen and triglycerides is promoted (Khan A H, Pessin J E. Insulin regulation of glucose uptake: a complex interplay of intracellular signalling pathways. Diabetologia. 2002 November;45(1 ):1475-83). However, insulin also has an important role with respect to the inhibition of muscle protein catabolism, or inhibition of protein breakdown, (Volpi E and Wolfe B. Insulin and Protein Metabolism. In: Handbook of Physiology, L. Jefferson and A. Cherrington editors. New York: Oxford, 2001, p. 735-757). Insulin drivers are substances which may enhance or promote the normal activity of endogenous insulin. For example, enhancing or promoting the action of insulin may promote the development of muscle through several mechanisms. For example, through the promotion of the uptake of glucose by muscle cells, insulin supplies muscles with a source of fuel. Additionally, insulin has also been shown to stimulate the uptake of amino acids by muscle cells and further stimulate protein synthesis (Biolo G, Declan Fleming R Y, Wolfe R R. Physiologic hyperinsulinemia stimulates protein synthesis and enhances transport of selected amino acids in human skeletal muscle. J Clin Invest. 1995 February;95(2):811-9), particularly following exercise (Biolo G, Williams B D, Fleming R Y, Wolfe R R. Insulin action on muscle protein kinetics and amino acid transport during recovery after resistance exercise. Diabetes. 1999 May;48(5):949-57).

Moreover, insulin has been shown to inhibit protein degradation (Hamel FG, Bennett R G, Harmon K S, Duckworth W C. Insulin inhibition of proteasome activity in intact cells. Biochem Biophys Res Commun. 1997 May 29;234(3):671-4; Bennett R G, Hamel F G, Duckworth W C. Insulin inhibits the ubiquitin-dependent degrading activity of the 26S proteasome. Endocrinology. 2000 July;141(7):2508-17) which may lead to muscle loss. The inhibition of proteolysis by insulin has experimentally been shown to be due to multiple mechanisms. First, insulin directly inhibits the catalytic activity of the proteasome by inhibiting its peptide-degrading action (Duckworth W C, Bennett R G, Hamel F G. A direct inhibitory effect of insulin on a cytosolic proteolytic complex containing insulin-degrading enzyme and multicatalytic proteinase. J Biol Chem. 1994 October 7;269(40):24575-80). Second, insulin has been shown to interfere with and downregulate the ATP-dependent ubiquitin (Ub) pathway (Price S R, Bailey J L, Wang X, Jurkovitz C, England B K, Ding X, Phillips L S, Mitch W E. Muscle wasting in insulinopenic rats results from activation of the ATP-dependent, ubiquitin-proteasome proteolytic pathway by a mechanism including gene transcription. J Clin Invest. 1996 October 15;98(8):1703-8; Mitch W E, Bailey J L, Wang X, Jurkovitz C, Newby D, Price S R. Evaluation of signals activating ubiquitin-proteasome proteolysis in a model of muscle wasting. Am J Physiol. 1999 May;276(5 Pt 1):C1132-8). Third, insulin reduces the expression of MAFbx, a muscle-specific Ub-ligase required for muscle atrophy. Sacheck J M, Ohtsuka A, McLary S C, Goldberg A L. IGF-I stimulates muscle growth by suppressing protein breakdown and expression of atrophy-related ubiquitin ligases, atrogin-1 and MuRF1. Am J Physiol Endocrinol Metab. 2004 October;287(4):E591-601). In combination, the aforementioned mechanisms may lead to the inhibition of muscle catabolism via insulin dependent mechanisms.

It would therefore be advantageous to enhance or promote the activity of insulin for the general preservation or growth of skeletal muscle, particularly for individuals involved in physical activity or training. In such individuals, the breakdown of muscle protein stimulated by exercise (Rennie M J, Edwards R H, Krywawych S, Davies C T, Halliday D, Waterlow J C, Millward D J. Effect of exercise on protein turnover in man. Clin Sci (Lond). 1981 November;61(5):627-39) is of concern and it is desired to be advantageously avoided or minimized.

There are a number of potential processes or variables which may be affected by insulin drivers in order to enhance or promote the activity of insulin. These steps may include, but not be limited to: insulin sensitivity, insulin secretion, binding of insulin to an insulin receptor, and transporter function (e.g. GLUT4 translocation).

### SUMMARY OF THE INVENTION

The foregoing needs and other needs and objectives that will become apparent for the following description are achieved in the present invention which comprises, according to various embodiments, a dietary supplement for enhancing or promoting the natural action of insulin or any individual aspect or combination of aspects thereof. The dietary supplement is advantageous for individuals wishing to enhance the growth of skeletal muscle, reduce the loss of skeletal muscle or increase the energy supply to active muscles. The composition of the present invention comprises at least D-Pinitol and Leucine or derivatives thereof. Furthermore, the present invention may comprise at least one of Alpha Lipoic Acid, Glucomannan, D-Myo-lnositol, Guar Gum, Taurine or derivative thereof, Ketoisocaproic Acid or derivative thereof, Alpha-Ketoglutarate or derivative thereof, and a source of Peptide C12.

The present invention also provides, by consumption of the dietary supplement by an individual, e.g., a human or an animal, a method for enhancing or promoting the activity of insulin, enhancing skeletal muscle growth, reducing skeletal muscle loss, and increasing the energy supply to skeletal muscle.

### DETAILED DESCRIPTION OF THE INVENTION

The present invention, according to various embodiments, is directed to enhancing or promoting the natural activity of endogenous insulin within the body of an individual, e.g., a human or an animal, enhancing the growth of skeletal muscle, reducing the loss of skeletal muscle via protein degradation, and increasing the energy supply of active muscles.

### D-Pinitol

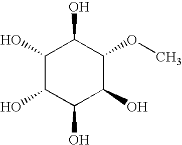

Pinitol (CAS Registry No 484-684) is the active principle found in Bougainvillea spectabilis, a traditional anti-diabetic plant. It is known to be in many legumes and pine wood. Futhermore, Pinitol may also be derived from the processing of soybeans (Nordin P. Preferential Leaching of Pinitol from Soybeans during Imbibition. Plant Physiol. 1984 Oct;76(2):313-315). It has been demonstrated to have insulin-like effects in animal models of diabetes (Bates S H, Jones R B, Bailey C J. Insulin-like effect of pinitol. Br J Pharmacol. 2000 August;130(8):1944-8). When Pinitol has been isolated from soybeans, it has been clinically shown to reduce risk factors in cardiovascular disease (Kim J I, Kim J C, Kang M J, Lee M S, Kim J J, Cha I J. Effects of pinitol isolated from soybeans on glycaemic control and cardiovascular risk factors in Korean patients with type II diabetes mellitus: a randomized controlled study. Eur J Clin Nutr. 2005 March;59(3):456-8) and postprandial blood glucose levels in patients with diabetes (Kang M J, Kim J I, Yoon S Y, Kim J C, Cha I J. Pinitol from soybeans reduces postprandial blood glucose in patients with type 2 diabetes mellitus. J Med Food. 2006 Summer;9(2):182-6).

Of particular interest to the invention, Pinitol has also been reported to increase creatine retention in muscle (Rasmussen C, Greenwood M, Kreider R, Earnest C, Almada A, Greenhaff P. Influence of D-Pinitol on whole body creatine retention. Medicine and Science in Sport and Exercise. 33(5): S204, 2001; Greenwood M, Kreider R B, Rasmussen C, Almada A L, Earnest C P. D-Pinitol augments whole body creatine retention in man. J Exerc Physiolonline 2001; 4:41-47).

In an embodiment of the present invention, which is set forth in greater detail in the example below, the supplemental composition includes D-Pinitol or a derivative thereof. A serving of the supplemental composition may include from about 0.5 μg to about 10 μg of D-Pinitol. The preferred dosage of a serving of the supplemental composition comprises about 2 μg of D-Pinitol.

### L-Leucine

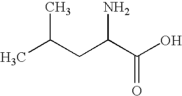

Leucine (CAS Registry No 328-39-2) is one of three branched chain amino acids and is important for skeletal muscle protein synthesis. Leucine is known to stimulate the mammalian target of rapamycin (mTOR) pathway (Anthony J C, Yoshizawa F, Anthony T G, Vary T C, Jefferson L S, Kimball S R. Leucine stimulates translation initiation in skeletal muscle of postabsorptive rats via a rapamycin-sensitive pathway. J Nutr. 2000 October;130(10):2413-9), a factor extremely important in muscle growth. mTOR is a complex protein pathway containing several regulatory sites as well as sites for interaction with multiple other proteins which acts to integrate signals pertaining to the energetic status of the cell as well as environmental stimuli to control protein synthesis, protein breakdown and, therefore, cell growth (Hay N, Sonenberg N. Upstream and downstream of mTOR. Genes Dev. 2004 Aug 15;18(16):1926-45). The mTOR kinase controls the translation machinery, in response to amino acids and growth factors, such as insulin. Leucine appears to stimulate mTOR in a manner similar to insulin, however acting via different mechanism.

The ingestion of Leucine combined with protein and carbohydrates has been shown to stimulate a reduction in protein breakdown and an increase in skeletal muscle protein synthesis to a greater degree than protein plus carbohydrate and carbohydrate alone (Koopman R, Wagenmakers A J, Manders R J, Zorenc A H, Senden J M, Gorselink M, Keizer H A, van Loon L J. Combined ingestion of protein and free leucine with carbohydrate increases postexercise muscle protein synthesis in vivo in male subjects. Am J Physiol Endocrinol Metab. 2005 April;288(4):E645-53). Furthermore, oral administration of Leucine has been shown to stimulate protein synthesis in the skeletal muscle of rats (Crozier S J, Kimball S R, Emmert S W, Anthony J C, Jefferson L S. Oral leucine administration stimulates protein synthesis in rat skeletal muscle. J Nutr. 2005 March;135(3):376-82). This may be mediated by the ability of Leucine to phosphorylate eIF4G, thereby facilitating the formation of a complex of eIF4G with the initiation factor eIF4E, a complex necessary for protein sysnthesis (Bolster D R, Vary T C, Kimball S R, Jefferson L S. Leucine regulates translation initiation in rat skeletal muscle via enhanced eIF4G phosphorylation. J Nutr. 2004 July;134(7):1704-10).

In an embodiment of the present invention, which is set forth in greater detail in the example below, the supplemental composition includes Leucine or derivatives thereof. A serving of the supplemental composition may include from about 1 μg to about 15 μg of Leucine or derivatives thereof. The preferred dosage of a serving of the supplemental composition comprises about 5 μg of Leucine or derivatives thereof.

### Alpha Lipoic Acid

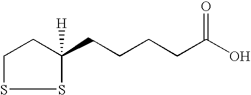

Alpha Lipoic Acid (CAS Registry No 6246-4) is an enzyme found in the cellular energy-producing structures, the mitochondria. Additionally, Alpha Lipoic Acid works in synergy with vitamins C and E as an antioxidant in both water- and fat- soluble environments.

In rats supplemented with Alpha Lipoic Acid the negative age-related changes in mitochondrial function, accumulated oxidative damage and the metabolic rate were all improved (Hagen T M, Ingersoll R T, Lykkesfeldt J, Liu J, Wehr C M, Vinarsky V, Bartholomew J C, Ames A B. (R)-alpha-lipoic acid-supplemented old rats have improved mitochondrial function, decreased oxidative damage, and increased metabolic rate. FASEB J. 1999 February;13(2):411-8). The antioxidant activity of Alpha Lipoic Acid is likely involved in the prevention of cell death due to oxidative stress (Arivazhagan P, Juliet P, Panneerselvam C. Effect of dl-alpha-lipoic acid on the status of lipid peroxidation and antioxidants in aged rats. Pharmacol Res. 2000 March;41 (3):299-303) and likely increased mitochondrial membrane permeability. Possibly related to these effects, Alpha Lipoic Acid has been linked to a beneficial increase in high-density lipoproteins (Wollin S D, Wang Y, Kubow S, Jones P J. Effects of a medium chain triglyceride oil mixture and alpha-lipoic acid diet on body composition, antioxidant status, and plasma lipid levels in the Golden Syrian hamster. J Nutr Biochem. 2004 July;15(7):402-10). Furthermore, Alpha Lipoic Acid appears to possess a dual action related to hunger and fl-oxidation of fat. First, the activity of AMP-activated protein kinase, which acts as an energy sensor in the hypothalamus, is reduced by Alpha Lipoic Acid in rodents, this results in a profound weight loss by reducing food intake and enhancing energy expenditure (Kim M S, Park J Y, Namkoong C, Jang P G, Ryu J W, Song H S, Yun J Y, Namgoong I S, Ha J, Park I S, Lee I K, Viollet B, Youn J H, Lee H K, Lee K U. Anti-obesity effects of alpha-lipoic acid mediated by suppression of hypothalamic AMP-activated protein kinase. Nat Med. 2004 July;10(7):727-33). Second, Alpha Lipoic Acid increases Uncoupling Protein-1 in rodent adipocytes while increasing AMP-activated protein kinase in skeletal muscle cells and increasing glucose uptake and energy expenditure (Lee W J, Koh E H, Won J C, Kim M S, Park J Y, Lee K U. Obesity: the role of hypothalamic AMP-activated protein kinase in body weight regulation. Int J Biochem Cell Biol. 2005 November;37(11):2254-9). Thus Alpha Lipoic Acid seemingly has different effects in different tissues. However, in adipocytes or muscle cells Alpha Lipoic Acid increases fatty acid oxidation, leading to an increase in energy expenditure and concomitant decreases in body weight and food intake.

U.S. Pat. Nos. 6,136,339 and 6,620,425 disclose compositions and methods for enhancing an athlete's muscle size or strength using a combination of Creatine, Alpha Lipoic Acid and optionally dextrose, to be taken mixed with water daily following exercise.

In an embodiment of the present invention, which is set forth in greater detail in the example below, the supplemental composition may include Alpha Lipoic Acid or derivatives thereof. A serving of the supplemental composition may include from about 1 μg to about 30 μg of Alpha Lipoic Acid or derivatives thereof. The preferred dosage of a serving of the supplemental composition comprises about 15 μg of Alpha Lipoic Acid or derivatives thereof.

### Glucomannan

Glucomannan is a polysaccharide composed of long chains of simple sugars, primarily mannose and glucose. It is classified as a soluble fiber. Glucomannan can be obtained from several plants, however, the primary source is an Asian origin plant named Amorphophallus Konjac.

Glucomannan has been shown to improve glycemic control and offer potential treatment for type 2 diabetes (Vuksan V, Jenkins D J, Spadafora P, Sievenpiper J L, Owen R, Vidgen E, Brighenti F, Josse R, Leiter L A, Bruce-Thompson C. Konjac-mannan (glucomannan) improves glycemia and other associated risk factors for coronary heart disease in type 2 diabetes. A randomized controlled metabolic trial. Diabetes Care. 1999 June;22(6):913-9; Chen H L, Sheu W H, Tai T S, Liaw Y P, Chen Y C. Konjac supplement alleviated hypercholesterolemia and hyperglycemia in type 2 diabetic subjects—a randomized double-blind trial. J Am Coll Nutr. 2003 February;22(1):3642) and improve insulin resistance in humans (Vuksan V, Sievenpiper J L, Owen R, Swilley J A, Spadafora P, Jenkins D J, Vidgen E, Brighenti F, Josse R G, Leiter L A, Xu Z, Novokmet R. Beneficial effects of viscous dietary fiber from Konjac-mannan in subjects with the insulin resistance syndrome: results of a controlled metabolic trial. Diabetes Care. 2000 January;23(1):9-14).

In an embodiment of the present invention, which is set forth in greater detail in the example below, the supplemental composition may include Glucomannan. A serving of the supplemental composition may include from about 0.01 g to about 0.5 g of Glucomannan. The preferred dosage of a serving of the supplemental composition comprises about 0.09 g of Glucomannan.

### D-myo-inositol

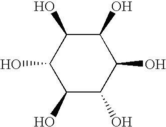

D-myo-inositol (CAS Registry No 87-89-8) is a distinct isomer of inositol, which is vital to a diverse range of biological processes. D-myo-inositol has the effect of priming the secretion of insulin (Hoy M, Berggren P O, Gromada J. Involvement of protein kinase C-epsilon in inositol hexakisphosphate-induced exocytosis in mouse pancreatic beta-cells. J Biol Chem. 2003 September 12;278(37):35168-71; Barker C J, Berggren P O. Inositol hexakisphosphate and beta-cell stimulus-secretion coupling. Anticancer Res. 1999 September-October;19(5A):373741), an effect that is dependent on protein kinase C (Efanov A M, Zaitsev S V, Berggren P O. Inositol hexakisphosphate stimulates non-Ca2+-mediated and primes Ca2+-mediated exocytosis of insulin by activation of protein kinase C. Proc Natl Acad Sci U S A. 1997 April 29;94(9):4435-9). Furthermore, the glomerular cells of diabetic rats display an increased transport of D-myo-inositol compared to non-diabetic rats (Whiteside C I, Thompson J C. Upregulation of D-myo-inositol transport in diabetic rat glomerular cells. Am J Physiol. 1992 March;262(3 Pt 1):E301-6) which may explain why supplementation may help individuals with diabetes.

In an embodiment of the present invention, which is set forth in greater detail in the example below, the supplemental composition may include D-myo-inositol. A serving of the supplemental composition may include from about 0.05 g to about 1 g of D-myo-inositol. The preferred dosage of a serving of the supplemental composition comprises about 0.2 g of D-myo-inositol.

### Guar Gum

Guar gum (CAS Registry No 9000-30-0) is a water soluble polysaccharide obtained from the guar bean (Cyamopsis tetragonoloba). In animal models of diabetes, guar gum has been shown to improve insulin sensitivity (Cameron-Smith D, Habito R, Barnett M, Collier G R. Dietary guar gum improves insulin sensitivity in streptozotocin-induced diabetic rats. J Nutr. 1997 February;127(2):359-64). In healthy rats, Guar Gum has the effect of improving insulin response (Prieto P G, Cancelas J, Villanueva-Penacarrillo M L, Malaisse W J, Valverde I. Short-term and Long-term Effects of Guar on Postprandial Plasma Glucose, Insulin and Glucagon-like Peptide 1 Concentration in Healthy Rats. Horm Metab Res. 2006 June;38(6):397-404). Supplementation with Guar Gum in humans allowed for better control of diabetes (Aro A, Uusitupa M, Voutilainen E, Hersio K, Korhonen T, Siitonen O. Improved diabetic control and hypocholesterolaemic effect induced by long-term dietary supplementation with guar gum in type 2 (insulin-independent) diabetes. Diabetologia. 1981 July;21(1):29-33) and reduced the associated cardiovascular risks (Uusitupa M, Tuomilehto J, Karttunen P, Wolf E. Long term effects of guar gum on metabolic control, serum cholesterol and blood pressure levels in type 2 (non-insulin-dependent) diabetic patients with high blood pressure. Ann Clin Res. 1984;16 Suppl 43:126-31) as shown by long-term studies. Additionally, the addition of low amounts of Guar Gum to the diet of humans has been shown to improve insulin sensitivity (Tagliaferro V, Cassader M, Bozzo C, Pisu E, Bruno A, Marena S, Cavallo-Perin P, Cravero L, Pagano G. Moderate guar-gum addition to usual diet improves peripheral sensitivity to insulin and lipaemic profile in NIDDM. Diabete Metab. 1985 December;11(6):380-5). In healthy humans insulin sensitivity is also improved (Landin K, Holm G, Tengborn L, Smith U. Guar gum improves insulin sensitivity, blood lipids, blood pressure, and fibrinolysis in healthy men. Am J Clin Nutr. 1992 December;56(6):1061-5).

The benefits conferred by Guar Gum are believed to be, at least partially, due to slowed glucose absorption (Russo A, Stevens J E, Wilson T, Wells F, Tonkin A, Horowitz M, Jones K L. Guar attenuates fall in postprandial blood pressure and slows gastric emptying of oral glucose in type 2 diabetes. Dig Dis Sci. 2003 July;48(7):1221-9), thereby improving insulin sensitivity.

In an embodiment of the present invention, which is set forth in greater detail in the example below, the supplemental composition may include Guar Gum. A serving of the supplemental composition may include from about 0.1 g to about 1.5 g of Guar Gum. The preferred dosage of a serving of the supplemental composition comprises about 0.5 g of Guar Gum.

### Taurine

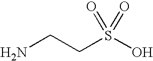

Taurine (CAS Registry No 107-35-7) is an amino acid found primarily in nerve and muscle tissue. Taurine is generally considered to be a conditionally-essential amino acid, wherein it is only required under certain circumstances. Although not utilized for protein synthesis, Taurine is found in free-form or in some small peptides.

Moreover, the accumulation of Taurine within cells is mediated by a high affinity sodium-dependent transporter (Ramamoorthy S, Leibach F H, Mahesh VB, Han H, Yang-Feng T, Blakely R D, Ganapathy V. Functional characterization and chromosomal localization of a cloned taurine transporter from human placenta. Biochem J. 1994 June 15;300 (Pt 3):893-900). The expression of this Taurine transporter is induced by a differentiation program of myocytes (muscle cells) (Uozumi Y, Ito T, Hoshino Y, Mohri T, Maeda M, Takahashi K, Fujio Y, Azuma J. Myogenic differentiation induces taurine transporter in association with taurine-mediated cytoprotection in skeletal muscles. Biochem J. 2006 March 15;394(Pt 3):699-706). Exercise-induced hormones also activate the Taurine transporter (Park S H, Lee H, Park T. Cortisol and IGF-1 synergistically up-regulate taurine transport by the rat skeletal muscle cell line, L6. Biofactors. 2004;21(1-4):403-6). Genetically modified mice lacking the Taurine transporter have depleted Taurine levels in all muscle and have impaired skeletal muscle function (Warskulat U, Flogel U, Jacoby C, Hartwig H G, Thewissen M, Merx M W, Molojavyi A, Heller-Stilb B, Schrader J, Haussinger D. Taurine transporter knockout depletes muscle taurine levels and results in severe skeletal muscle impairment but leaves cardiac function uncompromised. FASEB J. 2004 March; 18(3):577-9)

One of the main roles of Taurine is the regulation of fluid balance. Contracting muscles also release Taurine (Cuisinier C, Michotte De Welle J, Verbeeck R K, Poortmans J R, Ward R, Sturbois X, Francaux M. Role of taurine in osmoregulation during endurance exercise. Eur J Appl Physiol. 2002 October;87(6):489-95) as it has also been shown to modulate the contractile function of mammalian skeletal muscle (Bakker A J, Berg H M. Effect of taurine on sarcoplasmic reticulum function and force in skinned fast-twitch skeletal muscle fibres of the rat. J Physiol. 2002 January 1;538(Pt 1):185-94). In rats, the Taurine concentration in muscle decreases as a result of exercise (Matsuzaki Y, Miyazaki T, Miyakawa S, Bouscarel B, Ikegami T, Tanaka N. Decreased taurine concentration in skeletal muscles after exercise for various durations. Med Sci Sports Exerc. 2002 May;34(5):793-7) and oral supplementation with Taurine can maintain the concentration of Taurine in muscle and prolong exercise performance (Miyazaki T, Matsuzaki Y, Ikegami T, Miyakawa S, Doy M, Tanaka N, Bouscarel B. Optimal and effective oral dose of taurine to prolong exercise performance in rat. Amino Acids. 2004 December;27(3-4):291-8; Yatabe Y, Miyakawa S, Miyazaki T, Matsuzaki Y, Ochiai N. Effects of taurine administration in rat skeletal muscles on exercise. J Orthop Sci. 2003;8(3):415-9).

In a model of spontaneous diabetes, Taurine supplemented rats had improved insulin sensitivity (Nakaya Y, Minami A, Harada N, Sakamoto S, Niwa Y, Ohnaka M. Taurine improves insulin sensitivity in the Otsuka Long-Evans Tokushima Fatty rat, a model of spontaneous type 2 diabetes. Am J Clin Nutr. 2000 January;71(1):54-8). Furthermore, Taurine improves glucose metabolism in insulin resistant rats (Nandhini A T, Anuradha C V. Taurine modulates kallikrein activity and glucose metabolism in insulin resistant rats. Amino Acids. 2002;22(1):27-38). Supplementation with Taurine has been shown to reduce exercise-induced oxidative damage and enhance recovery (Zhang M, Izumi I, Kagamimori S, Sokejima S, Yamagami T, Liu Z, Qi B. Role of taurine supplementation to prevent exercise-induced oxidative stress in healthy young men. Amino Acids. 2004 March;26(2):203-7).

In an embodiment of the present invention, which is set forth in greater detail in the example below, the supplemental composition may include Taurine or derivatives thereof. A serving of the supplemental composition may include from about 0.02 g to about 1 g of Taurine or derivatives thereof. The preferred dosage of a serving of the supplemental composition comprises about 0.1 g of Taurine or derivatives thereof.

### Taurine Ketoisocaproic Acid

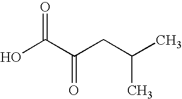

Taurine Ketoisocaporic Acid serves in the present invention as an additional source of Taurine in addition to providing Alpha-ketoisocaproate (CAS Registry No 816-66-0). Alpha-ketoisocaproate is a keto acid of the branched chain amino acid, Leucine. Moreover, Ketoisocaproic acid is known to stimulate insulin release (Heissig H, Urban K A, Hastedt K, Zunkler B J, Panten U. Mechanism of the insulin-releasing action of alpha-ketoisocaproate and related alpha-keto acid anions. Mol Pharmacol. 2005 October;68(4):1097-105; Rabaglia M E, Gray-Keller M P, Frey B L, Shortreed M R, Smith L M, Attie A D. Alpha-Ketoisocaproate-induced hypersecretion of insulin by islets from diabetes-susceptible mice. Am J Physiol Endocrinol Metab. 2005 August;289(2):E218-24) and Ketoisocaproic Acid has been shown to reduce protein catabolism as well as prevent muscle loss (Stewart P M, Walser M, Drachman D B. Branched-chain ketoacids reduce muscle protein degradation in Duchenne muscular dystrophy. Muscle Nerve. 1982 March;5(3):197-201).

In an embodiment of the present invention, which is set forth in greater detail in the example below, the supplemental composition may include Ketoisocaproic Acid or derivatives thereof. A serving of the supplemental composition may include from about 0.02 μg to about 5 μg of Ketoisocaproic Acid or derivatives thereof. The preferred dosage of a serving of the supplemental composition comprises about 1 μg of Ketoisocaproic Acid or derivatives thereof.

### Taurine Alpha-Ketoglutarate

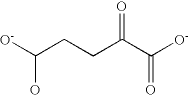

Taurine Alpha-Ketoglutarate serves as a source of Taurine as well as a source providing Alpha-Ketoglutarate. Alpha-Ketoglutarate (CAS Registry No 64-15-3) is an intermediate formed during the metabolism of glutamate. Alpha-Ketoglutarate also has a role in energy production via entry in to the tricarboxylic acid-cycle and oxidation to CO₂. Additionally, Alpha-Ketoglutarate is important for tissue healing (Aussel C, Coudray-Lucas C, Lasnier E, Cynober L, Ekindjian O G. alpha-Ketoglutarate uptake in human fibroblasts. Cell Biol Int. 1996 May;20(5):359-63), particularly in muscle (Wernerman J, Hammarqvist F, Vinnars E. Alpha-ketoglutarate and postoperative muscle catabolism. Lancet. 1990 March 24;335(8691):701-3) where Alpha-Ketoglutarate can preserve protein synthesis and prevent muscle loss (Hammarqvist F, Wernerman J, von der Decken A, Vinnars E. Alpha-ketoglutarate preserves protein synthesis and free glutamine in skeletal muscle after surgery. Surgery. 1991 January;109(1):28-36). Mutations in glutamate dehydrogenase, an enzyme involved in the formation of Alpha-Ketoglutarate, leads to the accumulation of Alpha-Ketoglutarate. This leads to chronic hyper-insulinemia which subsequently results in severe hypoglycemia (Stanley C A, Lieu Y K, Hsu B Y, Burlina A B, Greenberg C R, Hopwood N J, Perlman K, Rich B H, Zammarchi E, Poncz M. Hyperinsulinism and hyperammonemia in infants with regulatory mutations of the glutamate dehydrogenase gene. N Engl J Med. 1998 May 7;338(19):1352-7). It is further believed that the formation of Alpha-Ketoglutarate by glutamate dehydrogenase stimulates insulin secretion by supplying substrate for the tricarboxylic acid-cycle (Anno T, Uehara S, Katagiri H, Ohta Y, Ueda K, Mizuguchi H, Moriyama Y, Oka Y, Tanizawa Y. Overexpression of constitutively activated glutamate dehydrogenase induces insulin secretion through enhanced glutamate oxidation. Am J Physiol Endocrinol Metab. 2004 February;286(2):E280-5).

In an embodiment of the present invention, which is set forth in greater detail in the example below, the supplemental composition may include Alpha-Ketoglutarate or derivatives thereof. A serving of the supplemental composition may include from about 5 μg to about 30 μg of Alpha-Ketoglutarate or derivatives thereof. The preferred dosage of a serving of the supplemental composition comprises about 15 μg of Alpha-Ketoglutarate or derivatives thereof. Promoting the Activity of Insulin to Enhance Skeletal Muscle Growth Based on the aforementioned research, it has been determined by the inventors of the present invention that substances which enhance or promote the activity of insulin, or similar such insulin drivers, would be of benefit in terms of the natural role of insulin. In an embodiment of the present invention, insulin drivers may promote the growth of skeletal muscle in response to resistance exercise. It is an object of the present invention that insulin drivers may promote the maintenance of skeletal muscle during periods of inactivity. Another object of the present invention it that insulin drivers may promote the uptake of glucose by skeletal muscle to provide ample energy during strenuous or prolonged physical activity. From consideration of this specification and the foregoing example, other embodiments may be obvious to those skilled in the art.

Furthermore, Leucine, and other branch chain amino acids are known to activate the mTOR pathway of protein synthesis and decrease protein catabolism. Therefore, in terms of muscle building, it has been determined by the inventors of the present invention that it may be advantageous to provide compounds such a D-pinitol and derivatives thereof, which are known to mimic the actions of insulin and compounds such Leucine which are known be initiators of protein synthesis and inhibitors of protein catabolism. Through the concomitant administration of insulin-mimicking compounds and initiators of protein synthesis pathways and inhibitors of protein catabolism, it has been determined by the inventors of the present invention that nutrients can be more readily taken into the cell to facilitate the synthesis of protein commenced by known initiators of the mTOR pathway. Moreover, both insulin and Leucine act to inhibit the catabolism of protein. Therefore, it has been determined by the inventors of the present invention that the co-administration of insulin-mimicking substances and initiators of the mTOR pathway is advantageous in terms of muscle building.

According to various embodiments of the present invention, the dietary supplement may be consumed in any form. For instance, the dosage form of the diet supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention is as a powdered beverage mix. The dietary supplement may be provided alone or as part of a larger composition.

Furthermore, the dosage form of the dietary supplement may be provided in accordance with customary processing techniques for herbal and dietary supplements in any of the forms mentioned above. Additionally, the diet supplement set forth in the example embodiments herein may contain any appropriate number and type of excipients, as is well known in the art.

Preferably, the dietary supplement is consumed by an individual in accordance with the following method: As a diet supplement, a serving of said dietary supplement may be taken with an 8 oz. glass of water at least one time daily wherein each serving is comprised of approximately 1 g of said dietary supplement. Said dietary supplement may be consumed approximately 30 to 60 minutes before each meal, preferably in the morning and afternoon as well as after a workout. In this manner, the dietary supplement may enhance the growth of skeletal muscle, reduce the loss of skeletal muscle or increase the energy supply to active muscles of an individual, e.g. a human or an animal for an extended period of time, e.g., all day.

The present diet supplement or those similarly envisioned by one of skill in the art, may be utilized in compositions and methods to enhance the growth of skeletal muscle, reduce the loss of skeletal muscle or increase the energy supply to active muscles in an individual, e.g. a human or an animal.

Although the following example illustrates the practice of the present invention in one of its embodiments, the example should not be construed as limiting the scope of the invention. Other embodiments will be apparent to one of skill in the art from consideration of the specifications and example.

### EXAMPLE 1

The ingredients of the dietary supplement, supplied in dry powder form, may be mixed with 8 ounces of water for consumption. The composition of the dietary supplement includes: D-Pinitol (2 μg), L-Leucine (3 μg), Leucine methyl ester HCl (1 μg), Leucine ethyl ester HCl (1 μg), Alpha Lipoic Acid (15 μg), Glucomannan (0.09 g), D-Myo-lnositol (0.2 g), Guar Gum (0.5 g), Taurine (0.05 g), Taurine Ketoisocaproic Acid (0.025 g), Taurine Alpha-Ketoglutarate (0.025 g) and Peptide C12 (1 μg). The dietary supplement may be consumed two to three times daily and prior to exercise.

In the foregoing specification, the invention has been described with specific embodiments thereof, however, it will be evident that various modifications and changes may be made thereto without departing from the broader spirit and scope of the invention.

What is claimed is:

1. A composition comprising D-Pinitol and Leucine or derivatives thereof.

2. The composition of claim 1, further comprising Alpha Lipoic Acid.

3. The composition of claim 1, further comprising Glucomannan.

4. The composition of claim 1, further comprising D-Myo-lnositol.

5. A dietary supplement comprising about 2 μg of D-Pinitol and about 5 μg of Leucine or derivatives thereof.

6. The dietary supplement of claim 5, further comprising about 15 μg of Alpha Lipoic Acid.

7. The dietary supplement of claim 5, further comprising about 0.09 g of Glucomannan.

8. The dietary supplement of claim 5, further comprising about 0.2 g D-Myo-Inositol.

9. The dietary supplement of claim 5, further comprising:
about 0.5 g of Guar Gum;
about 0.05 g of Taurine;
about 0.025 g of Taurine Ketoisocaproic Acid;
about 0.025 g of Taurine Alpha-Ketoglutarate; and
about 1 μg of Peptide C12.

10. A method of enhancing or promoting the activity of insulin in a human or animal comprising at least the step of administering a composition comprising D-Pinitol and Leucine or derivatives thereof.

11. The method of claim 10, further comprising Alpha Lipoic Acid.

12. The method of claim 10, further comprising Glucomannan.

13. The method of claim 10, further comprising D-Myo-lnositol.

14. A method of increasing skeletal muscle growth, reducing skeletal muscle loss, and increasing the energy supply to skeletal muscle comprising at least the step of administering a composition comprising D-Pinitol and Leucine or derivatives thereof.

15. The method of claim 14, further comprising Alpha Lipoic Acid.

16. The method of claim 14, further comprsing Glucomannan.

17. The method of claim 14, further comprising D-Myo-lnositol.
